(19) United States
(12) Patent Application Publication
(10) Pub. No.: US 2008/0140179 A1
(43) Pub. Date: Jun. 12, 2008

(54) Apparatus and method for minimizing flow disturbances in a stented region of a lumen

(76) Inventor: John F. Ladisa, Milwaukee, WI (US)

Correspondence Address: RYAN KROMHOLZ & MANION, S.C., POST OFFICE BOX 26618, MILWAUKEE, WI, 53226, US

(21) Appl. No.: 12/001,645

(22) Filed: Dec. 12, 2007

Related U.S. Application Data
(60) Provisional application No. 60/874,428, filed on Dec. 12, 2006.

(51) Int. Cl. A61F 2/84; A61F 2/82

(52) U.S. Cl. 623/1.11; 623/1.34; 623/1.38; 623/1.42; 623/1.49

(57) ABSTRACT

A stent according to the present invention incorporates an intraluminal scaffold for initial relief of stenoses or for providing support within bodily lumens, such as blood vessels, of children and adults. The scaffold minimizes flow disturbances in a stented region of a bodily lumen by the use of a strut having a transitional lumen surface, thereby limiting the potential for the development of neointimal hyperplasia and subsequent restenosis, and lessening the potential for early and late thrombosis formation and dislodgement. Any stent for use in any application can be initially manufactured with the strut shape of the current invention. Alternatively, the shape of the struts of existing stents can be modified during a post-processing procedure after fabrication thereby making the invention applicable to all stent designs presently available.

### RELATED APPLICATIONS

This application claims the benefit of U.S. Provisional Patent Application Ser. No. 60/874,428, filed 12 Dec. 2006, and entitled “Stent with Specialized Strut Shape.”

### BACKGROUND OF THE INVENTION

Stent implantation is an accepted clinical way of restoring patency and fluid flow distal to luminal stenoses. Unfortunately, excessive restenosis, often initiated by neointimal hyperplasia, limits the success of vascular stents in approximately thirty percent of the patient population. The number of patients experiencing restenosis may be higher for certain patient populations such as diabetics, where the instance has been reported to be as high as forty-two percent. Drug-eluting stents are now being used with increasing frequency as they have been shown to reduce rates of restenosis for several years after stent implantation. However, recent reviews have suggested that drug-eluting stents may simply delay restenosis, and may not facilitate healing of the intima that is damaged by prior vascular disease or the stent implantation procedure, as once thought. Thus, restenosis rates with drug-eluting stents may ultimately be similar to bare metal stents, albeit time-shifted. Moreover, drug-eluting technology may not be applicable to all patient populations, such as diabetics, for example, and locations within the vasculature, such as bifurcations. Stent geometry may be an important predictor of neointimal hyperplasia and restenosis rates tend to vary with stent design. Stent deployment usually causes damage to the lumen into which it is deployed. After such damage, the frictional forces acting on the wall of the lumen as a result of flowing fluid, also known as the wall shear stress, may mediate cellular proliferation that may lead to neointimal hyperplasia.

Inadequate healing associated with drug-eluting stents may be due to incomplete coverage of the stent struts by endothelial cells thereby making the vessel more prone to early and late thrombosis. This late thrombosis is defined as the presence of a platelet-rich thrombus encompassing more than 25% of the lumen beyond 30 days after stent implantation. Recent clinical findings indicate that 50% of patients who experience late thrombosis will die of a myocardial infarction if blood flow through the stented regions causes the thrombus to dislodge and embolize in the downstream vasculature.

Therefore, the art of stenting a bodily lumen would benefit from an improved stent structure and method of intrinsically reducing rates of restenosis and the potential for thrombus by minimizing flow disturbances through the stented region of a lumen.

### SUMMARY OF THE INVENTION

A stent according to the present invention preferably intrinsically reduces rates of restenosis and the potential for thrombus by minimizing flow disturbances through the stented region of a lumen by optimizing local flow patterns created by the stent. Such optimization minimizes indices of fluid dynamics implicated in neointimal hyperplasia and subsequent restenosis as well as the potential for thrombus formation and dislodgement. A stent according to the present invention is preferably a stent having an arrangement and stent-to-vessel ratio that optimizes scaffolding, but minimizes vessel damage and flow alterations through the stented region when optimally deployed.

### BRIEF DESCRIPTION OF THE DRAWINGS

FIG. 1 includes an elevational view of a prior intraluminal stent deployed in a lumen, two cross-sections taken at two longitudinal locations and associated graphs comparing normalized wall shear stress and neointimal hyperplasia.

FIG. 2A is an axial cross-section view of a strut of the stent of FIG. 1 deployed against a luminal wall.

FIG. 2B is a radial cross-section view of the strut of FIG. 2A deployed against the luminal wall.

FIG. 3 is a perspective view of an embodiment of a stent according to the present invention.

FIG. 4A is an axial cross-section view of a first embodiment of a stent strut according to the present invention deployed against a luminal wall.

FIG. 4B is a radial cross-section view of the strut of FIG. 4A deployed against the luminal wall.

FIG. 5A is an axial cross-section view of a second embodiment of a stent strut according to the present invention deployed against a luminal wall.

FIG. 5B is a radial cross-section view of the strut of FIG. 5A deployed against the luminal wall.

FIG. 6A is an axial cross-section view of a third embodiment of a stent strut according to the present invention deployed against a luminal wall.

FIG. 6B is a radial cross-section view of the strut of FIG. 6A deployed against the luminal wall.

FIG. 7A is an axial cross-section view of a fourth embodiment of a stent strut according to the present invention deployed against a luminal wall.

FIG. 7B is a radial cross-section view of the strut of FIG. 7A deployed against the luminal wall.

FIG. 8 provides two axial cross-sections of a stented area of a bodily lumen.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

Distributions of low wall shear stress established after stent implantation appear to modulate the development of neointimal hyperplasia in rabbit iliac arteries. As this neointimal hyperplasia occurs within the stented region, the lumen geometry and associated distributions of wall shear stress are temporally altered in a manner that progressively abolishes wall shear stress disparity. Geometric properties of an implanted stent, including the number, width and thickness of stent struts (i.e. intrastent linkages), as well as the severity of stent shortening, local scaffolding, and degree of luminal curvature created by the stent, may contribute to altered indices of wall shear stress associated with neointimal hyperplasia.

The number, thickness and width of the stent struts associated with an implanted stent can introduce potentially adverse distributions of wall shear stress leading to the development of neointimal hyperplasia and subsequent restenosis. Of these design parameters, the number and thickness of stent struts associated with the stent may have a greater impact on the development of neointimal hyperplasia and subsequent restenosis than the width of the stent struts. The thickness of stent struts generally determines the severity of protrusion into the flow domain of the fluid flowing through the bodily lumen, which, in turn, causes disruption of fluid flow. Similarly, by increasing the number of stent struts, greater scaffolding of the lumen is accomplished by limiting protrusion of the vessel wall through the stent strut and leads to increased axial and circumferential uniformity within the stented region. Conversely, increasing the width of struts adds material to the stent struts primarily in the direction of fluid flow and therefore is not associated with flow disturbances to the same extent as the other design considerations.

The geometry of the stented region after implantation influences patterns of blood flow through the stent that likely play an integral role in this process. In a recent review, five factors were listed as particularly influential to the potential for stent thrombosis after drug-eluting stent implantation including bifurcation lesions, poor strut apposition, placement of overlapping stents, use of longer stents than necessary and stent struts that infiltrated a necrotic core. These factors may also be detriments to the success of bare metal stents. Of these five factors the first four adversely influence fluid flow through the stented region of the vasculature. Collectively these findings indicate a potential desirability of minimizing altered blood flow and distributions of WSS in the vicinity of an implanted stent to reduce the potential for late thrombosis.

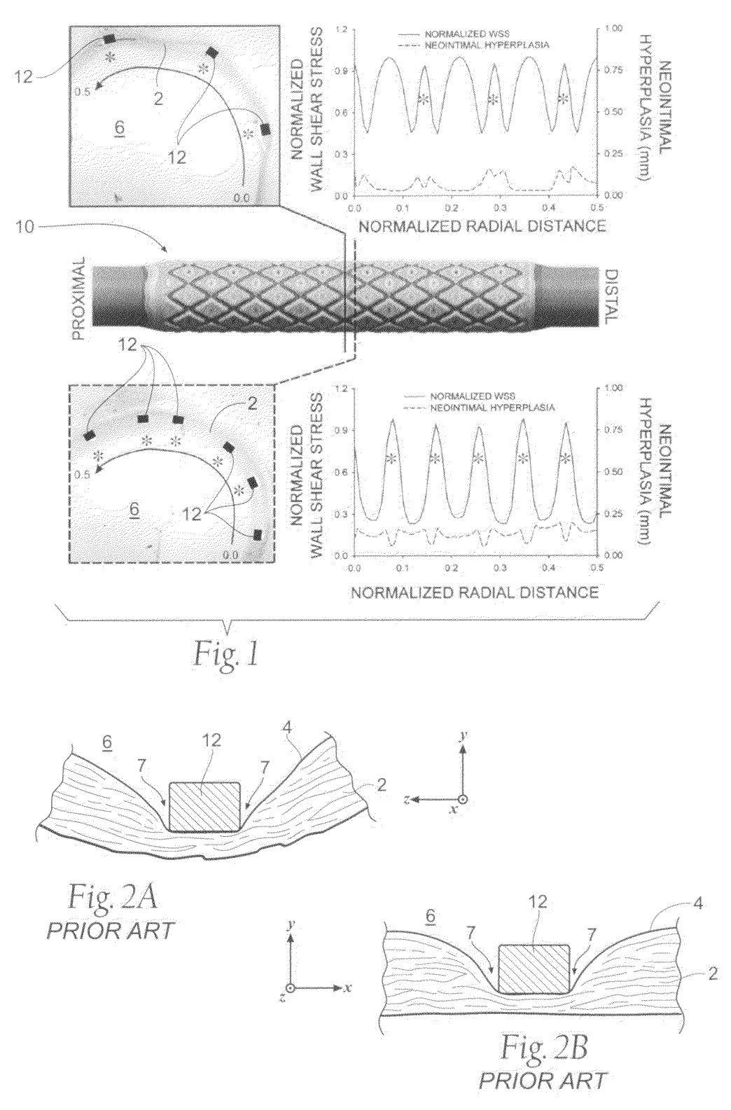

Turning now to the Figures, FIG. 1 depicts an implanted prior art stent 10 incorporating a strut configuration including struts 12 having generally rectangular cross-section. Generally, as can be seen, resulting distributions of wall shear stress after implantation and the spatial development of neointimal hyperplasia are associated. Neointimal hyperplasia is most pronounced in the regions of low wall shear stress introduced by the stent after implantation. FIGS. 2A and 2B are axial and radial cross-section views, respectively, of a strut 12 of the prior stent 10 having been deployed against a luminal wall 2 having an interior surface 4 that surrounds a flow domain 6, such as a lumen. The axes associated with FIGS. 2A and 2B are for reference only and fluid flow through the flow domain 6, such as blood through a blood vessel, may be presumed in the χ direction, as an example. Notice in FIGS. 2A and 2B, that after the stent 10 has been deployed and begins embedding into the vessel wall 2, the shape of the lumen 6 is at least partially dictated by the geometry of the stent strut 12. Implantation of the prior stent 10 provides supportive scaffolding for the vessel 2, but the abrupt transitions 7 from strut 12 to wall 2 generally create abrupt gradients of wall shear stress thereby causing the interior surface 4 of the lumen 2 to protrude into the flow domain 6 and disturb the axial and circumferential uniformity of the interior surface 4.

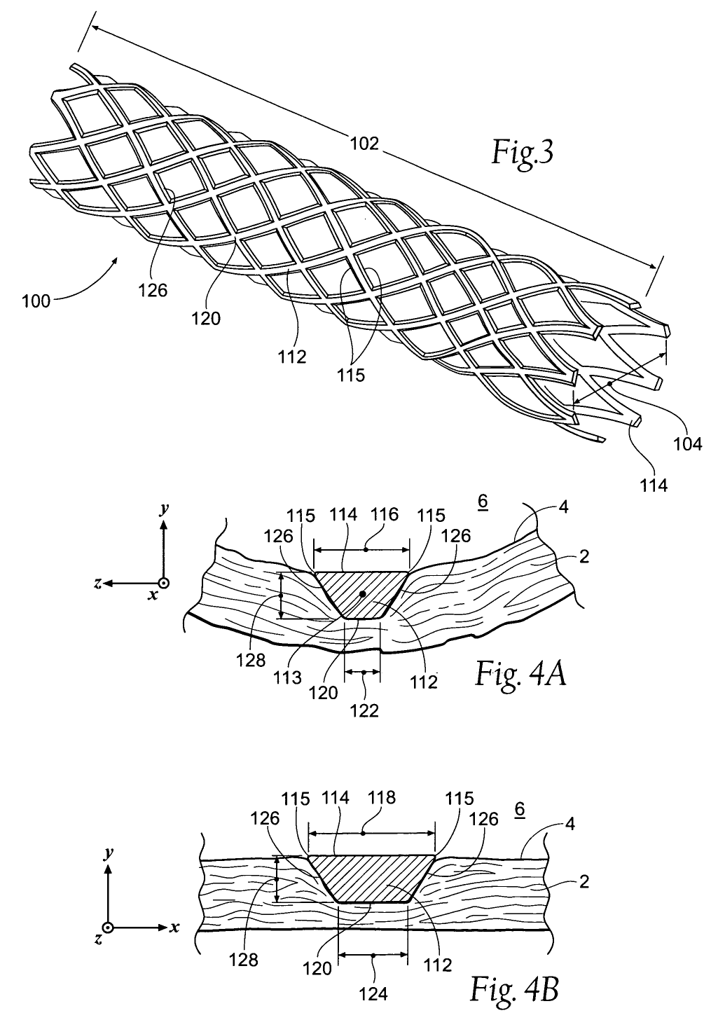

FIG. 3 provides a perspective view of an embodiment 100 of a stent according to the present invention. The embodiment 100 includes a plurality of interconnected struts 112, where the struts 112 are designed to limit protrusion into a flow domain. The struts 112 may be axially aligned and malleable, thereby facilitating flexibility and conformability to a variety of contours during positioning, deployment and after initial implantation. The stent 100 is provided at a desired length 102 and is expandable to at least a predetermined diameter 104. FIGS. 4A and 4B show axial and radial cross-section views, respectively, of a first embodiment 112 of a stent strut according to the present invention having been deployed against a luminal wall 2 having an interior surface 4 that surrounds a flow domain 6, such as a lumen. A stent strut is generally one of a plurality of interconnected support members that make up a stent. Each strut 112 has a length provided along a longitudinal axis 113, and further includes a flow surface 114 extending between flow surface edges 115. The flow surface edges 115 are preferably substantially or completely parallel to each other. The flow surface 114 has a flow surface circumferential width 116 and flow surface axial width 118. Generally opposed from the flow surface 114 is a scaffold surface 120 having a scaffold surface circumferential width 122 and scaffold surface axial width 124. The strut 112 also has transitional lateral side portions 126 that connect the flow surface 114 and the scaffold surface 120 through a radial thickness 128, which is measured orthogonally to the flow surface 114. The lateral side portions 126 preferably join the flow surface 114 along the flow surface edges 115. The radial thickness 128 of the strut 112 may be, for example, a maximum of 0.1 millimeters, or may be on the order of radial strut thicknesses associated with other commonly available stents.

A stent strut 112 having at least a portion formed according to the present invention includes the transitional lateral side portions 126 that provide a more gradual transition at the interface of the strut 112 and the luminal wall 2, such as a vessel wall, resulting in a smoother fluid flow surface in that portion of the flow domain 6. Generally, such transitional lateral side portions 126 may be formed by providing a desired ratio between the flow surface widths 116,118 and scaffold surface widths 122,124, respectively, and by sloping the lateral sides 126 from the flow surface edges 115 towards the scaffold surface 120. While the specific ratio between the flow surface widths 116,118 and scaffold surface widths 122,124, respectively, may be at least partially dictated by properties of the luminal wall 2, a flow surface width 116 may be preferably two to three times as wide as a corresponding scaffold surface width 122, for example. The circumferential widths 116,122 of at least a portion of a stent strut 112 may be related to the number of struts 112 provided around a given circumference of the stent 100. Additionally, axial widths 118,124 of at least a portion of a stent strut 112 may be related to the relative axial proximity of axially adjacent struts 112 within a given axial length of the stent 100. Both widths 116,122 and 118,124 may be selected or optimized in accordance with the embodiments shown but with the actual dimensions selected in order to provide the appropriate radial strength needed to maintain lumen patency upon implantation and resist collapsing.

The general goal of the transitional lateral side portions 126 is to provide a more gradual transition from stent 112 to wall 2 by allowing the wall 2 to more closely hug the stent 112, thereby at least reducing disturbances in the axial and circumferential uniformity of the interior surface 4 of the luminal wall 2 caused by the abrupt gradients of wall shear stress experienced with previous stents 10. As a result, flow turbulence and its associated neointimal hyperplasia and restenosis and the potential for thrombus formation and dislodgement are reduced. The shape of the stent struts 112, including, but not limited to, the circumferential and axial widths and thickness of the struts 112, is not necessary homogenous throughout the stent 100 or along any particular strut 112.

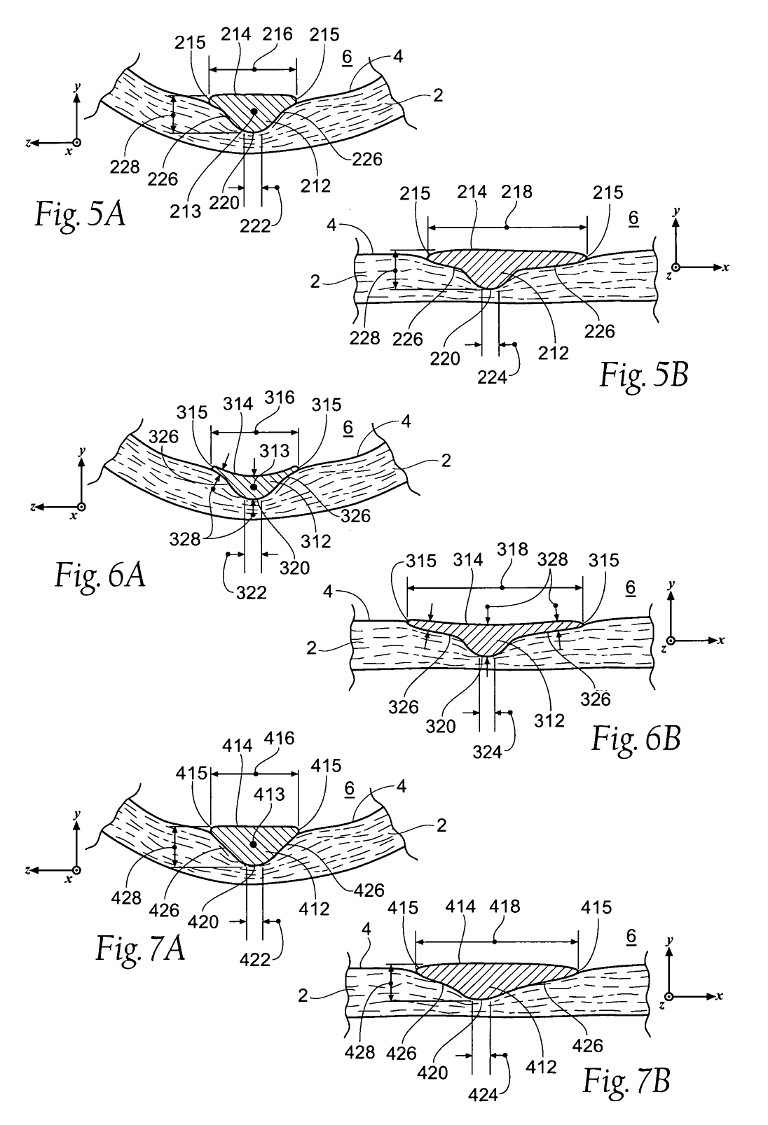

FIGS. 5A and 5B provide axial and circumferential views, respectively, of a second embodiment of a stent strut 212 according to the present invention, wherein similar reference numerals refer to similar structure of the first embodiment 112. This embodiment 212 includes a relatively flat flow surface 214, like the first embodiment 112, but also includes scalloped transitional lateral side portions 226 sloping towards a radiused scaffold surface 220.

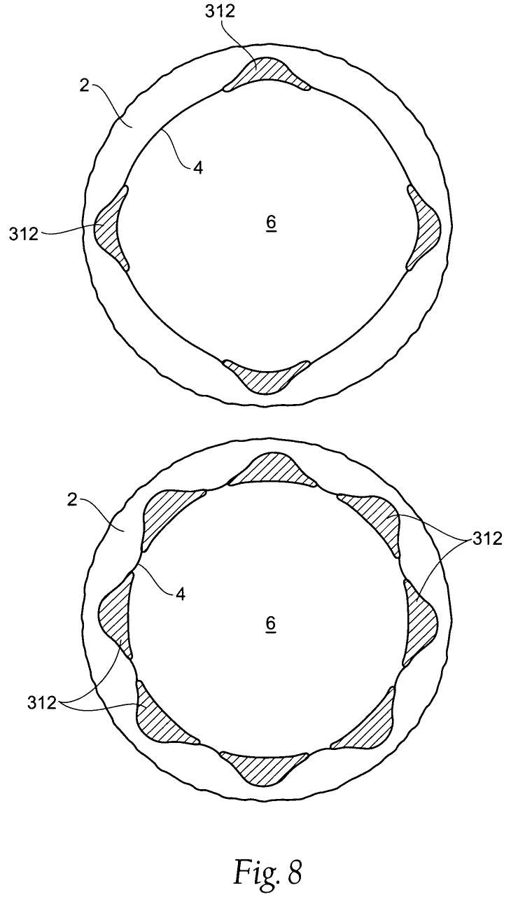

FIGS. 6A and 6B provide axial and circumferential views, respectively, of a third embodiment of a stent strut 312 according to the present invention, wherein similar reference numerals refer to similar structure of the first embodiment 112. In this embodiment 312, the flow surface 314 has been scalloped to further limit flow disturbances and facilitate a smooth transition between the stent strut 312 and the vessel wall 2. The radius or shape of this scalloped surface 314 may be related to the desired final deployment diameter of the stent and/or the total number of circumferential stent struts 312 at a given axial location of the stent. For example, a stent having four circumferential struts at a given axial location may have its struts 312 scalloped to a greater extent, i.e., have a smaller radius, than a stent having twice as many struts 312 at a given axial location, as depicted in FIG. 8. Additionally, the transitional lateral side portions 326 have been scalloped and the scaffold surface 320 has been smoothed to a radius. These design details may be related to the resiliency of the luminal wall 2.

FIGS. 7A and 7B provide axial and circumferential views, respectively, of a fourth embodiment of a stent strut 412 according to the present invention, wherein similar reference numerals refer to similar structure of the first embodiment 112. This embodiment 412 includes a relatively flat flow surface 414, like the first embodiment 112, but includes relatively flat transitional lateral side portions 426 sloping towards a radiused scaffold surface 420.

A stent according to the present invention may be initially manufactured with at least one strut according to the present invention. Alternatively, prefabricated stents having struts of an existing shape may be modified to include at least a portion of a strut according to the present invention by post-processing operations acting preferably on, but not limited to, the scaffold surface of at least one strut. Such post-processing operations may include, but are not limited to, laser cutting, grinding, microblasting, overblasting or electrical discharge machining. Further processing operations may include, but are not limited to electropolishing, etching or inducing an electrical charge to inhibit adverse tissue growth, cellular proliferation, neointimal hyperplasia, neoplastic growth and restenosis.

A stent according to the present invention may be constructed purely or from a composite of biocompatible materials including, but not limited to, the groups consisting of plastics (e.g. polymers), shape-memory (e.g. nickel-titanium) or deformable metals (e.g. 316L stainless steel or cobalt chromium) that facilitate the design attributes. Materials may be chosen to cause the stent to be sufficiently radiopaque, thereby facilitating its visualization during conventional angiography or other imaging procedures. Alternatively, or additionally, materials may be chosen to cause the stent to be sufficiently compatible with magnetic resonance imaging (MRI).

A stent according to the present invention may also be provided with drug-eluting, biocompatible, therapeutic agents or specialized processing procedures to prevent the adverse development of cellular proliferation, neointimal hyperplasia, neoplastic growth, restenosis, thrombus formation and dislodgement, or for any other reason. Potential drug-eluting biocompatible agents that could be added to the stent discussed in the present invention may consist of, but are not limited to, anticoagulants, immunosuppressants, antineoplastic agents, antithrombolytics, antimicrotubular agents, substances targeted against growth factors, fibrinolytics, glycoproteinIIb/IIIa inhibitors, antiplatelet substances, antiproliferation agents, chimeric monoclonal antibodies, antiproinflammatory cytokines and allograft rejection drugs. Any drug-eluting biocompatible therapeutic agent associated with a stent according to the present invention may be embedded as part of a bioabsorbable portion of such stent. The agent would thus be released over the bioabsorption period. Also, the agent may be contained in a coating of the stent that is released over time without degradation of any portion of the stent.

A stent according to the present invention may be created or manufactured by cutting stents similar in geometry, but having different strut widths, and then combining or layering them using a technique such as, but not limited to, complete laser or spot welding. Alternatively, or additionally, a stent according to the present invention may be created or manufactured by coining or otherwise forging a stent with rectangular strut cross sections in a cylindrical configuration, sheet configuration or other configuration after it has been laser cut or electrical discharge machined from a sheet or cylinder. Alternatively, or additionally, a stent according to the present invention may be created or manufactured by coining or otherwise forging a sheet or cylinder stent and then laser cutting or electrical discharge machining the stent shape from the coined or forged material. Alternatively, or additionally, a stent according to the present invention may be created or manufactured by cutting the stent pattern using a conventional 2-axis laser cutting system that is known to produce a cross sectional orientation that is opposite of that shown in FIGS. 7A and 7B. After cutting the entire stent, the stent can then be cut axially and rolled to produce the desired cross-sectional orientation. The stent may also be manufactured using thin-film fabrication techniques, such as photolithography. The stent may also be manufactured by laser cutting techniques whereby the laser, or its properties, is adjusted to achieve the desired shape.

A stent according to the present invention may be deployed to a predetermined diameter whereby scaffolding will be optimized and flow disturbances minimized. Such stent may be deployed using a conventional stent delivery balloon or a specialized delivery balloon that facilitates optimal conforming and transition from the stent struts to a luminal wall. A stent may be premounted on a delivery system or added to a separately packaged, preexisting delivery catheter. Alternatively, another specialized delivery device may be capable of determining when the deployment pressure to optimize the transition between stent struts and the vessel wall has been reached. A stent could also be deployed or implanted using a balloon catheter including sensors, that may be built into the walls of the balloon, that measure strain, pressure or change in pressure (directly or as a function of voltage change or some other measurand) inside the balloon beyond what is administered during inflation to determine when the optimal deployment as been obtained for a particular lumen.

A stent according to the present invention has a variety of uses. Such stent may be used in vascular and nonvascular regions corresponding, but not limited, to the cardiovascular system, respiratory system, digestive system, endocrine system, alimentary canal, renal system, and biliary tract. The stent may be used to treat vascular and nonvascular abnormalities including, but not limited to, coronary artery disease, peripheral vascular disease, coarctation of the aorta, peripheral pulmonary stenosis, homograft and conduit repairs after Tetralogy of Fallot, pulmonary vein stenosis, pyloric stenosis, intestinal atresia and stenosis, esophageal stricture, biliary stenosis, ischemic stroke, Takayasu's arteritis, superior vena cava syndrome, choanal atresia, ureteropelvic junction obstruction, and airway stenoses. Additionally, the implantation of such stent may be a method of preventing restenosis and repeat blockage or occlusion after percutaneous transluminal coronary angioplasty. The stent can be used as a method for re-establishing fluid flow in or repairing a vessel or bodily lumen.

A stent according to the present invention may be used by physicians including, but not limited to, interventional radiologists, endoscopists, interventional cardiologists, and general and vascular surgeons treating adults, neonates, children, adolescents, young adults, adults or elderly patients of either sex.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A support structure for placement in a bodily lumen, said structure comprising:
a plurality of interconnected support members, each of said support members having a length provided along a longitudinal axis, an inner flow surface extending along said length and between a pair of flow surface edges, said flow surface having a flow surface circumferential width measured orthogonally skew to said longitudinal axis, an outer scaffold surface having a scaffold surface circumferential width measured orthogonally skew to said longitudinal axis, a thickness measured orthogonally to said inner flow surface, and transitional lateral side portions extending between said flow surface and said scaffold surface;
wherein said flow surface circumferential width at a longitudinal location along said longitudinal axis of at least one support member is greater than said scaffold surface circumferential width at said longitudinal location.

2. A structure according to claim 1, wherein said structure is an intravascular stent.

3. A structure according to claim 1, said flow surface edges of said at least one support member being substantially parallel.

4. A structure according to claim 1, said structure comprising stainless steel.

5. A structure according to claim 1, said structure comprising a material that is radiopaque.

6. A structure according to claim 1, said structure being expandable to at least a predetermined diameter.

7. A structure according to claim 1, said structure comprising a material that is compatible with magnetic resonance imaging.

8. A structure according to claim 1, said structure further comprising a drug-eluting, biocompatible therapeutic agent coupled to at least a portion of one support member.

9. A structure according to claim 8, said portion of said support member, to which said agent is coupled, being bioabsorbable.

10. A structure according to claim 1, said thickness of said at least one support member adjacent said flow surface edges is less than fifty percent of said thickness at said longitudinal axis.

11. A structure according to claim 1, said at least one support member having a cross-section at said longitudinal location wherein said cross-section forms a mathematical concave set of points.

12. A structure according to claim 1, said at least one support member having a cross-section at said longitudinal location wherein said cross-section forms a mathematical convex set of points.

13. A structure according to claim 1, said longitudinal location having a flow surface circumferential width that is at least twice the magnitude of said scaffold surface circumferential width.

14. In a stent adapted for support of a luminal wall of a living body, said stent comprising a plurality of interconnected support members, said support members having a longitudinal axis, a scaffold surface adapted to interface said luminal wall, and a flow surface adapted to interface a lumen, the improvement comprising:
at least a portion along a length of said longitudinal axis of at least one of said interconnected support members having said flow surface being wider than said support surface.

15. An improvement according to claim 14, said portion further comprising transitional lateral side portions sloping from said flow surface edges towards said scaffold surface, forming an acute angle between said flow surface and said transitional lateral side portions.

16. An improvement according to claim 14, said improvement being formed by laser cutting.

17. A method of reducing flow disturbances in a fluid flow domain within a bodily lumen, said method comprising the steps of:
providing a support structure adapted for placement in a bodily lumen, said support structure comprising a plurality of interconnected support members, each of said support members having a length provided along a longitudinal axis, an inner flow surface extending along said length and between a pair of flow surface edges, said flow surface having a flow surface circumferential width measured orthogonally skew to said longitudinal axis, an outer scaffold surface having a scaffold surface circumferential width measured orthogonally skew to said longitudinal axis, a thickness measured orthogonally to said inner flow surface, and transitional lateral side portions extending between said flow surface and said scaffold surface, wherein said flow surface circumferential width at a longitudinal location along said longitudinal axis of at least one support member is greater than said scaffold surface circumferential width at said longitudinal location;
inserting said structure into a bodily lumen; and
expanding said structure to a desired diameter.

18. A method according to claim 17, further comprising the step of placing said structure on an inflatable balloon prior to inserting said structure into said bodily lumen and said expanding step being executed by said balloon acting upon said structure.

19. A method according to claim 17, further comprising the steps of forming an opening in a body of a patient and inserting said structure through said formed opening prior to inserting said structure into said bodily lumen.
